(19) United States
(12) Patent Application Publication
(10) Pub. No.: US 2003/0069622 A1
(43) Pub. Date: Apr. 10, 2003

(54) Method and apparatus for treatment of skin

(76) Inventor: Robert S. Backstein, Toronto (CA)

Correspondence Address: ZARLEY LAW FIRM P.L.C., CAPITAL SQUARE, 400 LOCUST, SUITE 200, DES MOINES, IA, 50309-2350, US

(21) Appl. No.: 10/254,111

(22) Filed: Sep. 25, 2002

(30) Foreign Application Priority Data
Oct. 9, 2001 (CA) 2,358,756

(51) Int. Cl. A61F 7/00

(52) U.S. Cl. 607/107; 607/109; 607/114

(57) ABSTRACT

The invention provides a method for the cosmetic treatment of skin comprising the use of chamber with an aperture to create an atmosphere of elevated temperature and exposing the skin area to said heated atmosphere to effect a mild superficial burn sufficient to remove surface skin and promote regeneration of the skin surface.

This invention relates to an improved method and apparatus for the treatment of skin. In particular, it relates to an improved method and apparatus for treatment for age related skin conditions to promote a cosmetic improvement resulting in a fresher more youthful skin, particularly in the facial area.

In recent times, the increased attention to cosmetic beauty combined with the technical advances in cosmetic surgery have lead to significant rise in the number of applications and the variety of techniques which are used to improve appearance, especially as it concerns skin condition.

These techniques include such methods as injections, resurfacing techniques, and plastic surgery, or variations of the foregoing.

While plastic surgeons and dermatologists have attempted to develop easy, safe and effective techniques to reduce facial wrinkles and improve skin tone, these techniques have generally been invasive and/or risky, and frequently require a degree of skill and coordination that is not always consistent.

Three of the main types of treatment include mechanical dermabrasion, chemical peeling, and laser resurfacing.

Dermabrasion involves the mechanical denuding or removing of the superficial layers of skin by using a series of coarse tips attached to a hand held rotor. This abrasive device is equivalent to “sanding” off the upper layers of the patient's facial skin and requires a steady hand and the knowledge of exactly how much pressure to apply to the treated area and for how long to apply this pressure to achieve the ideal depth of erosion. If too deep a burn is induced, the skin will heal with unsightly scarring which can also effect the function of eyelids, lips and other areas. Furthermore, mobile anatomical parts such as the eyelids can be injured if the operator is not careful to avoid contact with these parts. The abrasion process also produces airborne particles of the patient's blood which have been known to transmit communicable disease to others present at the procedure.

Chemical peeling involves the use of a noxious chemical such as trichloroacetic acid or phenol to induce a chemical burn to the patient's skin. Any deficiencies of a chemical peel include both operator error and the inherent toxicity of the chemicals used in the process. The operator must place a sufficient but not excessive amount of chemical on the skin to achieve the proper depth of burn. Furthermore, the operator must judge properly when to remove the chemical from the patient's skin to avoid deep scarring burn. Some chemicals such as phenol can also cause dangerous systemic side effects such as cardiac arrhythmias.

Laser resurfacing makes use of the heat generated by laser light to thermally burn the upper layers of skin. However, laser resurfacing is best performed by a physician with excellent manual dexterity a steady hand and good judgment in the amount of time to spend with the laser focussed on a given area of skin. Too much exposure to the laser light will result in unsightly scarring of the skin. Laser systems are extremely expensive to purchase and operate and the devices are very large, weighty and cumbersome to transport and use. The technique also requires that the user make careful application of the laser energy evenly and consisteulles to all areas of the shin.

Intense pulsed light, also known as radio frequency resurfacing, is a similar process but relatively newer procedure that requires the same skill and judgment (and subject to the same errors and risks) as laser treatments.

It has been known for some time that burns to the surface layers of skin, often accidentally induced by exposure to sunlight or to fire or flash explosions, can if they are of limited intensity, result in regeneration of new skin which is actually more youthful, tight and well toned. It is however important to control the depth or intensity of the burn by controlling the temperature and duration of exposure. It is also important in order to achieve the desired effect that the intensity of the treatment be uniform over the treated area.

It is therefore the purpose of this invention to provide a method and apparatus whereby a thermal treatment may be used to induce a superficial burn sufficient to cause a renewal or regeneration of the skin of the patient.

It is also the purpose of this invention to provide a method and apparatus whereby the application of the burn technique may be uniformly applied and controlled so as to provide a degree of consistency and safety in the application of the technique.

It is also the purpose of this invention to provide a regulated and automated application of the treatment without relying on the error prone judgment and skill of the operator.

It is also the purpose of this invention to provide a means for treatment over the entire area to be treated in a time which is dramatically reduced and is therefore less onerous and less expensive.

These objects and other advantages are sought to be achieved by the present invention which consists of an enclosure having an aperture in one side thereof and means associated with said enclosure to eject a stream of hot air into the inside of said enclosure.

The aperture is preferably designed to accommodate a portion of skin to be treated such as a face. Ideally, the device should have controls including an on/off switch and a thermostatic control to regulate the temperature of the air stream. Ideally the temperature controls will be fixed or adjustable at a very narrow range acceptable temperature to avoid operator error.

The invention therefore contemplates the use of a stream of heated air applied to an area of skin to create a superficial burn for the purpose of creating a renewed regenerated complexion. The treatment as contemplated depends on regulating the temperature and the duration of exposure of the skin to the heated air. The treatment is also facilitated by the controlled environment whereby the heated air is contained in an enclosure surrounding the treatment area so as to effectively control the temperature and duration of the treatment.

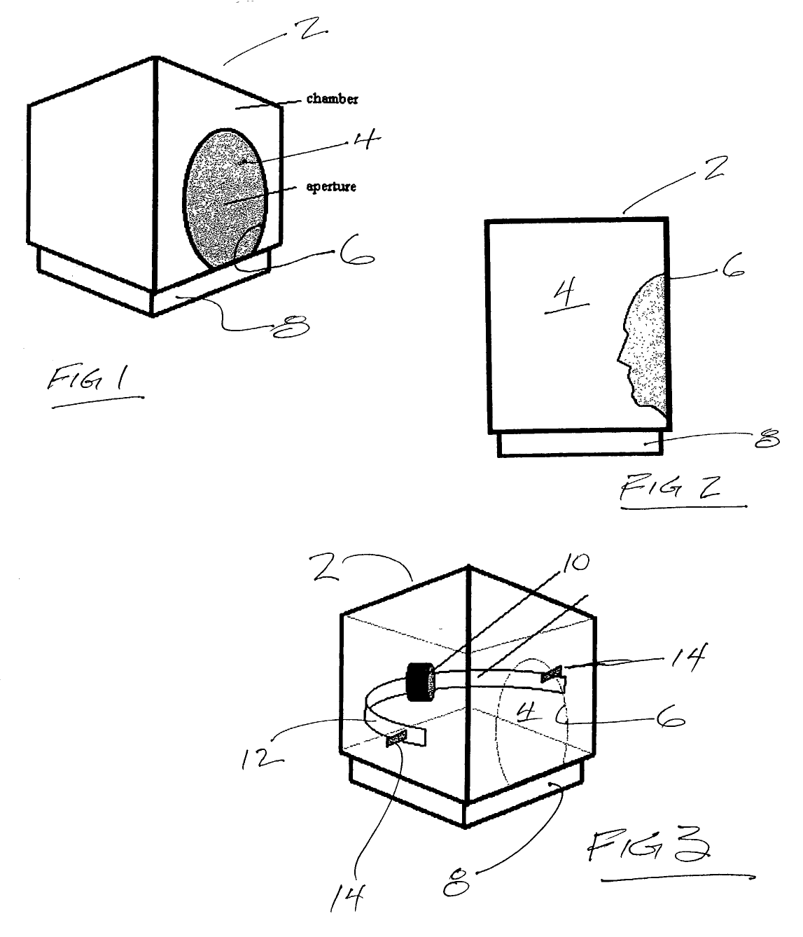

The invention may be better understood by a description of one embodiment thereof with reference to the attached drawings in which FIG. 1 is a perspective view of the enclosed apparatus employed in the present invention.

FIG. 2 is a vertical cross-section of the apparatus illustrated in FIG. 1.

FIG. 3 is a cut a way of the illustration of the internal structure of the apparatus in FIG. 1.

In the illustrated embodiment, referring to FIG. 1, a rectangular enclosure 2 provides an internal chamber 4 accessible through an aperture 6 which may be of any desirable size depending on the proposed application.

Also shown in FIG. 1 is a base 8 associated with the enclosure which contains apparatus for generating a stream of hot air. While the details of this mechanism is not subject of this application it is contemplated by the inventor that the necessary heat would be provided by an electrical resistence element and the stream of air would be generated by some sort of fan which may also be powered by the same electrical sources.

It is also contemplated although not illustrated that the mechanism 8 will be provided with the usual and appropriate controls. These would include an on/off switch and a thermostatic control adapted to variably regulate the temperature of the air stream introduced into the chamber 4. Preferably these controls should be either fixed or variable within narrow range of acceptable temperature to avoid operator error. The controls may also include timing means to avoid over-exposure to the selected or pre-set temperature.

It is anticipated that the atmospheric temperature in the chamber will be in the approximate range of 50 to 60° C. and the time of exposure of treatment will be relatively brief in the order of approximately 5 to 15 seconds. However, these variables are interrelated and will have to be determined by experience and experimentation.

The aperture 6 illustrated in FIG. 1 is somewhat oval and designed for the purpose of treating facial skin which may be exposed to the internal chamber as illustrated in FIG. 2. Obviously, the size of the aperture may be varied to accommodate people of different size or alternatively it may be provided with a surrounding flexible or elastic curtain which would enable the aperture to vary according to the size of the patient.

The object of the aperture is to allow a person's face (or other shin surface) to be introduced and exposed to the internal atmosphere of the chamber over an area that would extend from the neck to the hairline above the forehead and from the front of the ears on each side of the face. While other apertures may be designed to accommodate other shapes and sizes, the use for facial treatment is illustrated because it is the most common cosmetic object.

Of course it is not necessary that the enclosure or chamber be cubic. It may be any shape that provides an atmospheric chamber.

While the entry by which the hot air is introduced into the chamber is not illustrated in FIGS. 1 and 2 it is considered important that the hot air be circulated in such a way as to create a relatively even and consistent temperature throughout the chamber or at least in the area of the skin to be treated. This may be achieved by a number of inlet ports located in various positions to create circulation of air at a consistent temperature.

One embodiment of the invention is illustrated schematically in FIG. 3 in which a nozzle of an air blower 10 is mounted on a substantially semi-circular track 12 which will allow it to move from side to side while being aimed at the face or skin in the aperture. At the same time, the track 12 is mounted on pivots 14 which will allow the blower to be moved in an arc in the vertical direction so that it can be aimed from below, in front and above the face exposed in the aperture. These degrees of motion may be controlled via a keypad (electronically) or by manual buttons or handles on the chamber.

By means of the illustrated apparatus, a patient may be exposed to a superficial burn sufficient to eliminate the surface layers and cause regeneration.

The method of treatment will of course involve the necessary preliminary treatment to avoid pain to the patient. Once the exposed area is inserted in the aperture the machine can be activated to cause a stream of heated air to be directed at the patient's (facial) skin. The temperature of the air and the duration of the exposure will of course be determined by experience and professionally known parameters to effect the desirable degree of superficial burn.

It is however contemplated that because the heat is applied by a stream of air directed to the face in a broad stream as opposed to a localized focussed point, the application will be less subject to error and variation. Furthermore the application will be applied generally to the whole area as opposed to a focussed treatment which requires extensive movement to traverse the treated area.

It is additionally anticipated by directing a stream of heated air towards the treatment area the intensity of the application of this treatment will be diminished towards the outer edges of the treatment area so that there will be a less noticeable demarcation between the areas of treatment and the areas not treated.

The nozzle 10 can be changed to allow a much more focussed or constricted stream of air to treat a specific spot or localized are if desired.

Thus by means of the method and apparatus described above, it is possible to treat skin in a manner which avoids the toxicity of chemical peeling or the errors and variations involved in laser resurfacing or dermabrasion.

It will of course be realized that numerous modifications and variations of the illustrated embodiment may be employed without departing from the inventive concept herein.

1. Apparatus for the treatment of skin comprising:
an enclosure having a surrounding wall;
an aperture in said wall;
means to generate and inject heated atmosphere into said enclosure; and
means to regulate the temperature of said atmosphere as so to cause a mild superficial burn to the surface of skin.

2. Apparatus as claimed in claim 1, in which said atmosphere is air.

3. Apparatus as claimed in claim 1 including means to time the exposure of said heated atmosphere to said skin.

4. Apparatus as claimed in claim 2 including means to time the exposure of said heated atmosphere to said skin.

5. Apparatus as claimed in claim 1 in which the means to inject said atmosphere is a nozzle aimed in the direction of said aperture.

6. Apparatus as claimed in claim 2 in which the means to inject said atmosphere is a nozzle aimed in the direction of said aperture.

7. Apparatus as claimed in claim 3 in which the means to inject said atmosphere is a nozzle aimed in the direction of said aperture.

8. Apparatus as claimed in claim 5, in which said nozzle is movable along a horizontal path and a vertical path while being aimed in the direction of said aperture.

9. Apparatus as claimed in claim 6 in which said nozzle is movable along a horizontal path and a vertical path while being aimed in the direction of said aperture.

10. Apparatus as claimed in claim 7 in which said nozzle is movable along a horizontal path and a vertical path while being aimed in the direction of said aperture.

11. A method of treatment of skin comprising:
the use of a chamber having an aperture;
creating an atmosphere of elevated temperature in said chamber;
exposing said skin to said atmosphere through said aperture for a determined period of time sufficient to effect a mild superficial burn to the surface of said skin.
